(19) United States
(12) Patent Application Publication
(10) Pub. No.: US 2026/0144927 A1
(43) Pub. Date: May 28, 2026

(54) MINIATURIZED PATCH PUMP SYSTEM

(71) Applicant: UNOMEDICAL A/S, Lejre (DK)

(72) Inventor: Erik Othel-Jacobsen, Snekkersten (DK)

(21) Appl. No.: 19/338,313

(22) Filed: Sep. 24, 2025

Related U.S. Application Data
(63) Continuation of application No. 17/727,104, filed on Apr. 22, 2022, now Pat. No. 12,447,270.
Application No. 17/727,104 is a continuation of application No. PCT/EP2022/060652, filed on Apr. 22, 2022.
(60) Provisional application No. 63/178,816, filed on Apr. 23, 2021.

(51) Int. Cl. A61M 5/142; A61M 5/145; A61M 5/32; A61M 39/24

(52) U.S. Cl. CPC A61M 5/14248; A61M 5/14566; A61M 39/24; A61M 2005/3289; A61M 2205/0216; A61M 2205/125

(57) ABSTRACT

A patch pump system that has an electronic assembly, a reservoir for containing a fluid, and an inserter for selectively transitioning a needle from a retracted position to an extended position. The inserter provides a fluid channel that selectively fluidly couples a distal tip of the needle to the reservoir. The needle is at least partially deformed as it transitions from the retracted position to the extended position and the electronic assembly selectively distributes fluid from the reservoir to the distal tip of the needle.

### CROSS-REFERENCE TO RELATED DISCLOSURES

The present disclosure is a continuation of a continuation of U.S. application No. Ser. No. 17/727,104 filed Apr. 22, 2022, which is a continuation of International Application No. PCT/EP 2022/060652 filed on Apr. 22, 2022 and claims the benefit of U.S. Provisional Application No. 63/178,816 filed on Apr. 23, 2021, the contents of which are incorporated herein in entirety.

### FIELD OF THE DISCLOSURE

The present disclosure relates to a patch system for delivering a fluid subcutaneously to a user, and more specifically to a miniaturized patch system that has a needle insertion mechanism.

### BACKGROUND OF THE DISCLOSURE

Pump systems have been developed to provide a user with prolonged subcutaneous infusion of a fluid. Often, the pump system pumps fluid from a reservoir into tubing leading to an infusion set having a needle or cannula positioned subcutaneously in a user. The pump system may have corresponding electronics or mechanical components to provide a metered delivery of the fluid to the user. Often, the pump and reservoir are separated from the infusion set and the fluid from the reservoir is pumped to the infusion set through tubing.

### SUMMARY

One embodiment is a patch pump system that has an electronic assembly, a reservoir for containing a fluid, and an inserter for selectively transitioning a needle from a retracted position to an extended position, the inserter providing a fluid channel that selectively fluidly couples a distal tip of the needle to the reservoir. In this embedment, the needle is at least partially deformed as it transitions from the retracted position to the extended position and the electronic assembly selectively distributes fluid from the reservoir to the distal tip of the needle.

In one example of this embodiment, the reservoir has a base surface configured to be coupled to a user and the base surface defines a base plane. The inserter has an inner part that rotates about a rotation axis that is substantially parallel to the base plane to transition the needle from the retracted position to the extended position. One part of this example has a filter positioned between the reservoir and the distal tip of the needle. However, other parts of this example do not have a filter at all.

In examples having a filter, the filter at least partially filters one or more of phenol and meta-cresol residuals from fluid passing there through.

In one example of this embodiment, the patch system is less than forty-nine millimeters wide by thirty-nine millimeters deep and twelve millimeters thick.

In another example of this embodiment, the patch system is less than about forty-one millimeters wide by about thirty-six millimeters deep and about ten millimeters thick.

Another example of this embodiment includes a valve positioned along the fluid channel between the distal tip of the needle and the reservoir. In part of this example, the valve is a one-way valve allowing fluid to flow through the fluid channel from the reservoir to the distal end of the needle.

Yet another example of this embodiment has a filter positioned in-line with the fluid channel between the reservoir and the distal tip of the needle and a first one-way valve positioned along the fluid channel between the filter and the reservoir. An additional example may add a second one-way valve positioned along the fluid channel between the distal end of the needle and the filter.

In another example of this embodiment, the reservoir is removable from the electronic assembly and inserter.

In another example of this embodiment, the electronic assembly selectively transitions the needle between the retracted position to the extended position.

In another example of this embodiment, the electronic assembly selectively controls a motor to rotate the inserter to transition the needle between the retracted position to the extended position. As part of this example, the electronic assembly selectively controls an extension length of the needle. In another part of this example, the electronic assembly selectively controls an insertion angle of the needle.

Yet another example of this embodiment includes an inner part configured to selectively transition the needle from the retracted position to the extended position and an insertion angle component. In this example, as the inner part transitions from the retracted position to the extended position, the needle is at least partially deformed by the insertion angle component. In one part of this example, the needle is elastically deformed to fit within the inserter in the retracted position and deflects through contact with the insertion angle component as the needle transitions from the retracted position to the extended position. In another part of this example, the needle maintains a substantially linear configuration as it extends outside of the base.

Another embodiment of this disclosure is a patch pump system that has an electronic assembly, a reservoir for containing a fluid, and an inserter for selectively transitioning a needle from a retracted position to an extended position, the inserter providing a fluid channel that selectively fluidly couples a distal tip of the needle to the reservoir. In this embodiment, the patch pump system is less than forty-nine millimeters wide by thirty-nine millimeters deep and twelve millimeters thick.

In one example of this embodiment, the reservoir has a base surface configured to be coupled to a user, the base surface defining a base plane. The inserter extends the needle at least about four millimeters past the base plane in the extended position. In part of this example, the patch system is no greater than about forty-one millimeters wide by about thirty-six millimeters deep and about ten millimeters thick.

In another example of this embodiment, the reservoir is removable from the electronic assembly and inserter.

Yet another example of this embodiment has an inner part of the inserter configured to selectively transition the needle from the retracted position to the extended position and an insertion angle component. In this example as the inner part transitions from the retracted position to the extended position, the needle is at least partially deformed by the insertion angle component. In part of this example, the needle is elastically deformed to fit within the inserter in the retracted position and deflect through contact with the insertion angle component as the needle transitions from the retracted position to the extended position. In another part of this example, the needle maintains a substantially linear configuration as it extends outside of the base.

### BRIEF DESCRIPTION OF THE DRAWINGS

The above-mentioned aspects of the present disclosure and the manner of obtaining them will become more apparent and the disclosure itself will be better understood by reference to the following description of the embodiments of the disclosure, taken in conjunction with the accompanying drawings, wherein:

FIG. 1 is an elevated perspective view of a patch pump system;

FIG. 2 is a section view of the patch pump system of FIG. 1;

FIG. 3 is another section view of the patch pump system of FIG. 1;

FIG. 4a is a schematic view of an inserter of the patch pump system of FIG. 1 in the retracted configuration;

FIG. 4b is a schematic view of an inserter of the patch pump system of FIG. 1 in the extended configuration;

FIG. 4c is a schematic view of an inserter of the patch pump system of FIG. 1 having an adjustable insertion angle;

FIG. 5 is a schematic section view of the patch pump system of FIG. 1;

FIGS. 6a and 6b illustrate one embodiment of this disclosure utilizing a spring to extend a needle to an extended position;

FIGS. 7a-7c illustrate one embodiment wherein a reservoir is removable from a patch pump system; and

FIGS. 8a-8b illustrate one embodiment of a geared motor assembly for an inserter.

Corresponding reference numerals are used to indicate corresponding parts throughout the several views.

### DETAILED DESCRIPTION

The embodiments of the present disclosure described below are not intended to be exhaustive or to limit the disclosure to the precise forms in the following detailed description. Rather, the embodiments are chosen and described so that others skilled in the art may appreciate and understand the principles and practices of the present disclosure.

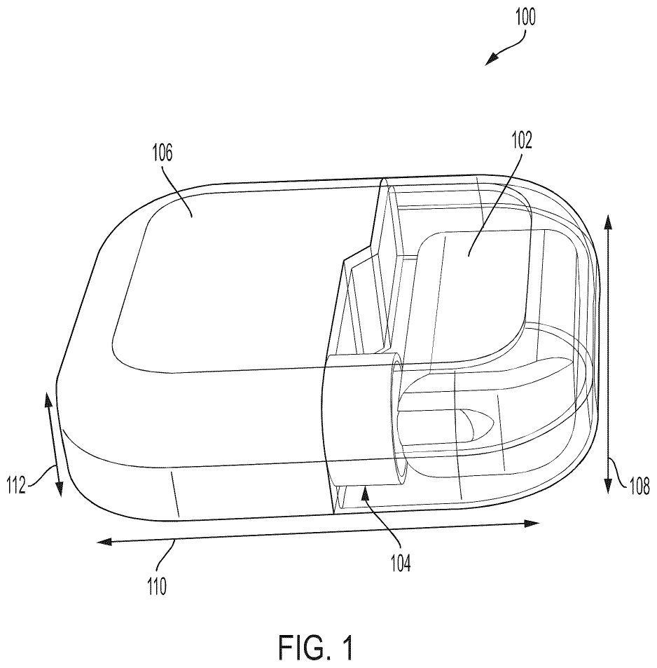

Referring to FIG. 1, one embodiment of a patch pump system 100 is illustrated. The patch pump system 100 may have a reservoir 102 that provides fluid to an infusion assembly having an inserter 104. Further, an electronic assembly 106 may be part of the patch pump system 100 to control the distribution of fluid from the reservoir 102 into components of the patch pump system 100 as discussed in more detail here.

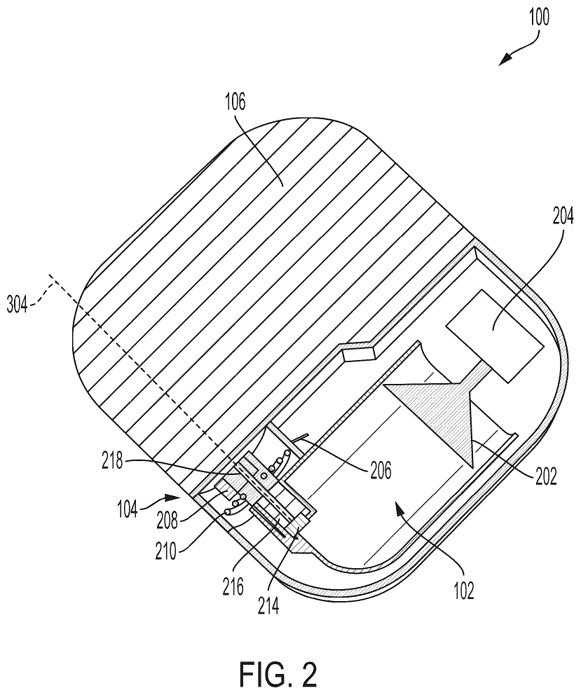

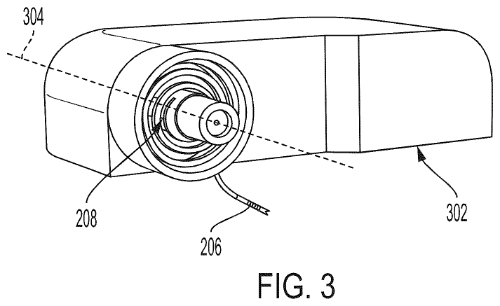

More specifically with reference to the section view of the patch pump system in FIGS. 2 and 3, the reservoir 102 may have a piston 202 or the like therein to selectively apply a pressure to the fluid in the reservoir 102. The piston 202 may be connected to a motor and gear assembly 204 to selectively actuate the piston 202 to move axially through the reservoir 102. This motor and gear assembly 204 may be powered and controlled by components from the electronic assembly 106. For example, the electronic assembly 106 may have a power source such as a battery therein and a controller having a processor and a memory unit. The controller may selectively power the motor and gear assembly 204 to move the piston 202 through the reservoir 102. In this configuration, the electronic assembly can dose any fluid in the reservoir 102 at predetermined rates or cycles.

While a motor and gear assembly is discussed herein, other embodiments considered herein may not have a motor and gear assembly but rather utilize a spring or the like to transition the piston 202. Alternatively, the motor may directly drive the piston 202 and not have a gear assembly at all in one embodiment considered herein.

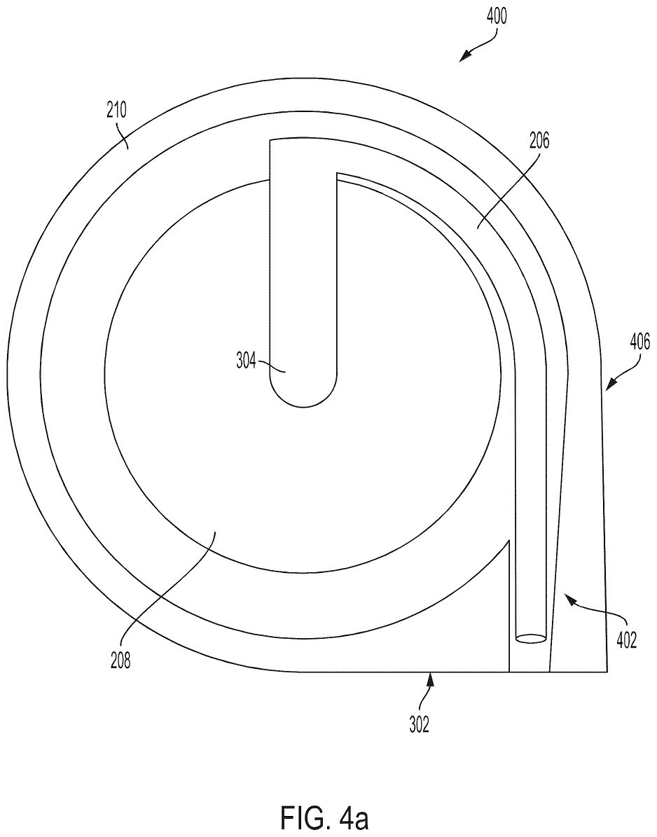

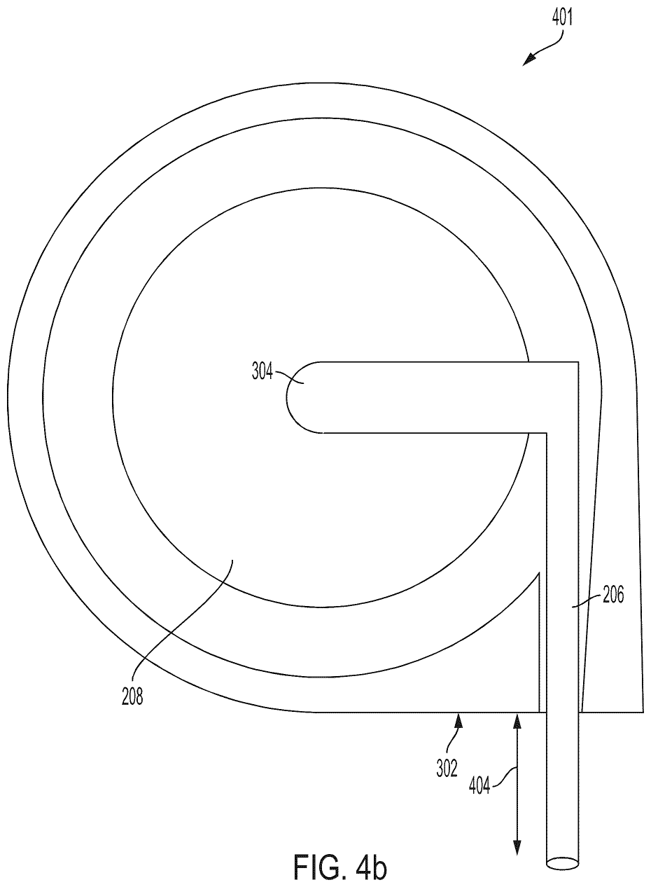

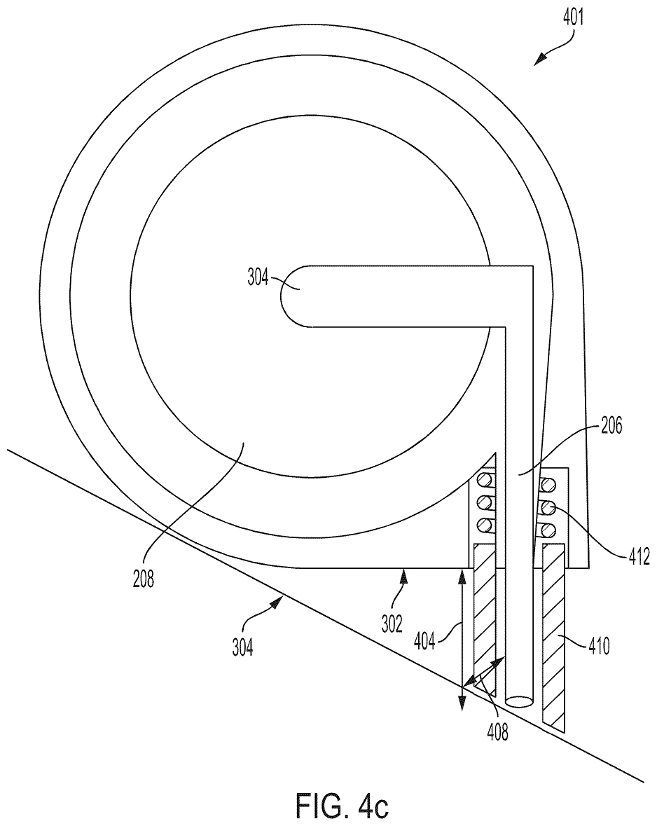

A portion of the reservoir 102 may be fluidly coupled to the inserter 104 to distribute fluid from the reservoir 102 out of a needle 206. The inserter 104 may selectively transition the needle 206 from a retracted position 400 (see FIG. 4a) wherein the needle is substantially housed within the inserter 104 to an extended position 401 (see FIG. 4b) wherein the needle 206 extends from a base plane along a base surface 302 of the patch pump system 100. More specifically, the inserter 104 may have an inner part 208 that rotates relative to a housing 210 of the inserter 104 about a rotation axis 304. The rotation axis 304 may be substantially parallel to the base plane of the base surface 302. Further, the inner part 208 may have a fluid channel 212 that is fluidly coupled to the reservoir 102. The fluid channel 212 directs fluid from the reservoir 102 through the inserter 104 and into the needle 206 to be exhausted out of a distal portion of the needle 206 when the piston 202 applies sufficient pressure to the fluid in the reservoir 102.

In one aspect of this disclosure, a valve 214 may be positioned at least partially between the fluid channel 212 and the reservoir 102. The valve 214 may be a one-way valve wherein fluid can pass from the reservoir 102 into the fluid channel 212 but not from the fluid channel 212 back into the reservoir 102. Among other things, this may prevent contaminated fluid from entering the reservoir 102. Further, the reservoir 102 may be removable from the remaining components of the patch pump system 100. In this configuration, when the fluid of the reservoir 102 in the patch pump system 100 is depleted, the depleted reservoir may be removed and a full reservoir may be fluidly coupled to the inserter 104 as a replacement.

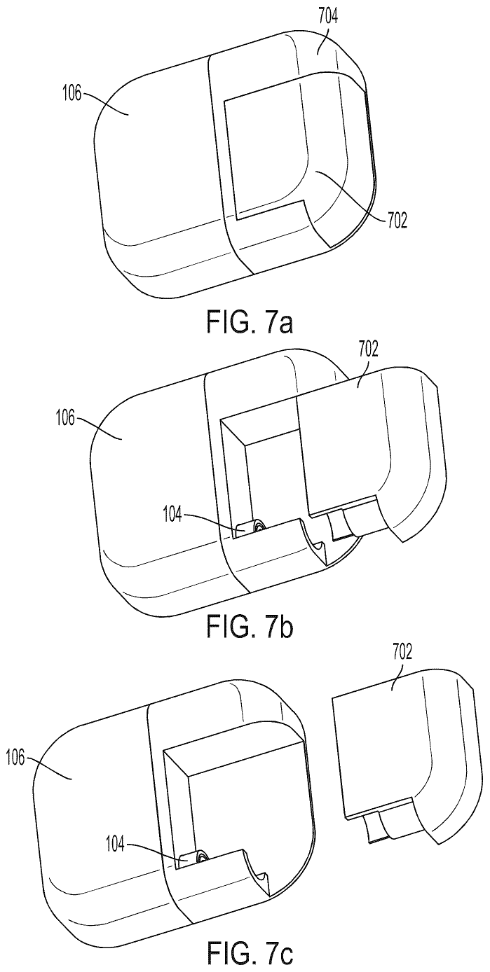

One embodiment of a removable reservoir 702 is illustrated in FIGS. 7a-7c. More specifically, the reservoir 702 may be removably coupled to a patch housing 704 such that it may slide into, and out of, fluid connection with the inserter 104. The patch housing 704 may define a receiving portion that corresponds with the reservoir 702 to allow the reservoir 702 to slide into, and out of fluid coupling with the inserter 104. In the fluidly coupled position, the reservoir 702 may have a latch or the like that locks the reservoir 702 into the fluidly coupled position with the inserter 104. However, when the reservoir 702 is depleted, the user may disengage the latch to remove the reservoir 702 from the patch housing 704. Then, a reservoir full of fluid may be subsequently coupled thereto for further use. In one aspect of this embodiment, a substantially circular seal may be positioned about the fluid channel 212 between the removable reservoir 702 and the inserter 104 to ensure the fluid channel 212 is substantially sealed at the interchange between the removable reservoir 702 and the inserter 104 when the removable reservoir 702 is latched to the patch housing 704.

In another aspect of this disclosure, a filter 216 may be positioned along the fluid channel 212. The filter 216 may filter phenol and meta-cresol residuals from the fluid flowing from the reservoir 102 to the needle 206 among other things. However, other types of filters for filter 216 are also contemplated herein.

The inserter 104 may also have a valve 218 positioned between the inner part 208 and the needle 206. Similar to the valve 214, valve 218 may also be a one-way valve that substantially prevents the backflow of fluid back into the reservoir 102 from the needle 206. Embodiments considered herein may have only one of the valves 214, 218 and not the other. Alternatively, one embodiment considered herein has both valves 214, 218 positioned as discussed and illustrated herein.

The inner part 208 may be coupled to the needle 206 such that rotation of the inner part 208 moves the needle 206 between the retracted position 400 and the extended position 401. The inner part 208 may be coupled to the needle 206 such that rotation of the inner part 208 relative to the housing 210 about the rotation axis 304 transitions the needle along an insertion guide channel 402 within an insertion angle component 406 before extending past the base surface 302. More specifically, the needle 206 may be formed of a material that has material properties such that the needle 206 can elastically deform within the inserter 104. The needle 206 may typically have a substantially linear configuration but the elastic properties of the needle 206 may allow the needle 206 to bend along the inner surface of the housing 210 when in the retracted position.

This arc-shaped elastic deformation of the needle 206 allows the inserter 104 and corresponding patch-pump system 100 to have a low profile (i.e., thickness 112) because it allows the needle 206 to achieve a desired extended depth 404 past the base surface 302 in the extended position 401 without requiring additional axial clearance for the needle to achieve the retracted position 400. In one example, the extended depth 404 may be about four millimeters. However, in other embodiments considered herein the extended depth 404 may be greater than four millimeters or less than four millimeters. Further, the degree to which the inner part 208 rotates may alter the extended depth 404 of the needle 206. In one aspect of this disclosure, the degree of rotation of the inner part 208 may be controlled by the electronic assembly 106 to alter the extended depth 404.

In other words, a substantially straight needle typically moves axially along a longitudinal axis of the needle to transition between the retracted and extended position. This requires enough axial space along the longitudinal axis of the needle to allow the needle to maintain the retracted position. By implementing the bending needle 206 configuration discussed herein, the needle 206 may partially bend along the housing 210 in the retracted position 400 to reduce the required thickness 112 of the patch-pump system 100 while still achieving the same extended depth 404 as the prior art designs.

The insertion guide channel 402 may be formed in the insertion angle component 406 that may be part of the housing 210. The insertion guide channel 402 may have any desired angular orientation relative to the base surface 302, which is intended to be positioned on the skin of the user. While the embodiments illustrated in FIGS. 4a and 4b primarily show the needle 206 extending substantially perpendicularly from the base surface 302, other embodiments may extend the needle 206 at about thirty degrees from the base surface 302. Alternatively, other embodiments considered herein may utilize an insertion guide channel 402 that directs the needle 206 to extend at an angle that is greater than thirty degrees but less than ninety degrees relative to the base surface 302. Further still, other embodiments may utilize an insertion guide channel 402 that directs the needle 206 to extend at an angle that is less than thirty degrees.

In one aspect of this disclosure illustrated in FIG. 4c, the inserter 104 may be pivotal within the patch pump system 100 to alter an insertion angle 408 of the needle 206 relative to the base surface 302 of the patch pump system 100. This allows the user or medical professional to select and insertion angle 408 that is ideal for the user. The needle 206 may have a sleeve 410 that is extendable along the needle 206 to ensure the needle 206 extends past the base surface 302 in a substantially linear configuration. The sleeve 410 may have a spring 412 positioned axially about the needle 206 between the sleeve 410 and the inserter 104. In this configuration, when the insertion angle 408 is about ninety degrees, the sleeve 410 may compress the spring 412 and be positioned substantially within a cavity of the inserter 104. However, when the insertion angle 408 is less than ninety degrees, the sleeve 410 may at least partially extend out of the cavity as the spring 412 provides a bias towards the base surface 302 to thereby ensure the sleeve 410 provides a channel for the needle 206 to be further inserted into the user.

In yet another aspect of this embodiment, the electronic assembly 106 may control the insertion angle 408. More specifically, the inserter 104 and housing 210 may be rotatable relative to the electronic assembly 106 to alter the insertion angle 408 as discussed herein. Further, a motor or the like may selectively rotate the inserter 104 through the housing 210 to alter the insertion angle 408. In this configuration, the electronic assembly 106 may selectively alter the insertion angle 408 by selectively rotating the inserter 104 via the motor.

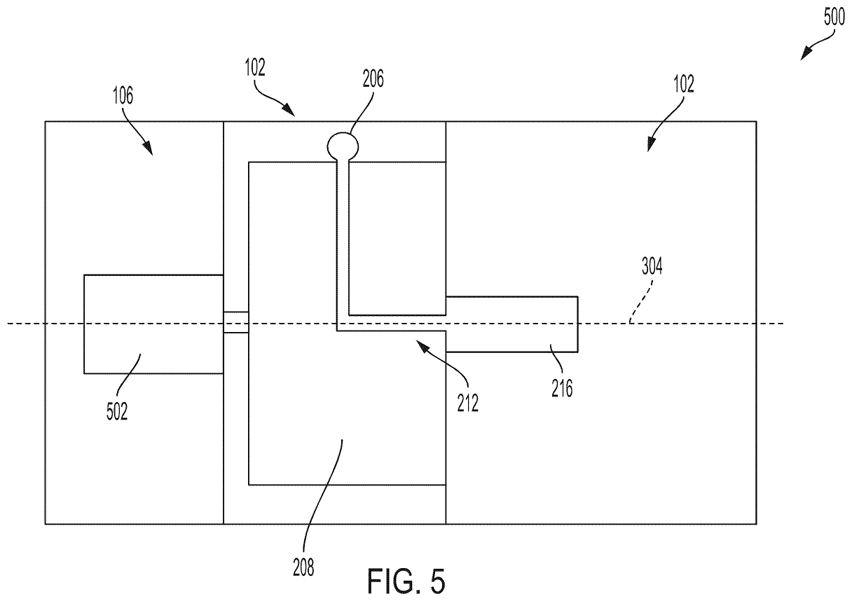

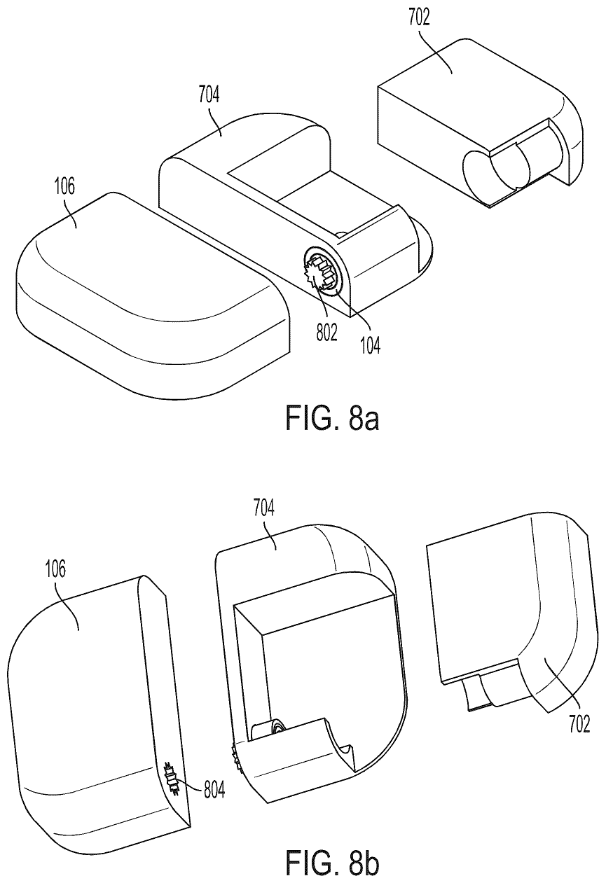

Referring now to FIG. 5, a schematic section view 500 of the patch-pump system 100 is illustrated. More specifically, a motor 502 is illustrated as part of the electronic assembly 106. The motor 502 has a shaft coupled to the inner part 208 of the inserter 104. Further, the motor 502 may be selectively powered by the controller of the electronic assembly 106. Alternatively, the motor 502 may be selectively powered by a user input. Regardless, the motor 502 may be powered to rotate the inner part 208 and the needle 206 between the retracted position 400 and the extended position 401. Further, in one embodiment contemplated herein the motor 502 may be coupled to a gear assembly 802, 804 (see FIGS. 8a and 8b) to selectively rotate the inner part 208. Further still, the gear assembly 802, 804 may be a planetary gear assembly or any other type of gear assembly known in the art.

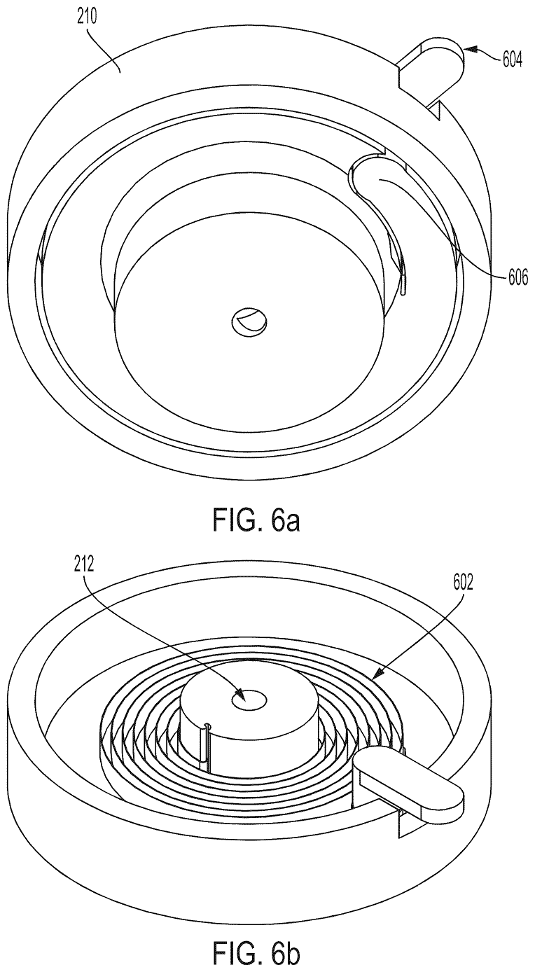

In another embodiment of this disclosure illustrated in FIGS. 6a and 6b, the inner part 208 may have a mechanical assembly configured to transition the needle 206 from the retracted position 400 to the extended position 401. More specifically, a spring 602 may be positioned to provide a bias on the inner part 308 relative to the housing 210 towards the extended position 401. The inner part 308 may be locked in the retracted position with an activator 604 and lock pin 606. The activator 604 may be engaged by a user to release the inner part 208 to rotate to the extended position 401 due to the bias applied by the spring 602.

The fluid channel 212 may transition to the inner part 208 along the rotation axis 304. With this configuration, the fluid channel 212 of the inner part 208 may remain fluidly coupled to the reservoir 102 through the fluid channel regardless of the angular orientation of the inner part 208 (i.e., regardless of whether the needle 206 is in the retracted position 400 or the extended position 401). In one aspect of this disclosure, a seal may be positioned between the inner part 208 and the reservoir 102. The seal may be circularly positioned about the fluid channel 212 at the interchange between the inner part 208 and the reservoir 102. Positioning the seal at this location may ensure the fluid in the fluid channel 212 is directed to the needle 206 from the reservoir 102.

In one aspect of this disclosure, the inserter 104 utilizing the bending needle 206 provides an overall patch pump system 100 that is significantly smaller than the prior art patch pump systems. More specifically, the patch pump system 100 may have a length 108, width 110, and thickness 112 that results in a miniature patch pump system compared to the prior art devices. In one example, the width 110 is less than fifty-six millimeters, the length 108 is less than forty-one millimeters, and the thickness is less than fourteen and one-half millimeters. In another example, the width 110 is less than forty-nine millimeters, the length 108 is less than thirty-nine millimetres, and the thickness is less than twelve millimeters. In yet another example, the width 110 is about forty-one millimeters, the length 108 is about thirty-six millimeters, and the thickness is about ten millimeters.

In use, the patch-pump system 100 may have an adhesive or the like on the base surface 302. Alternatively, the patch-pump system 100 may have an adhesive element that extends over at least a portion of the patch-pump system 100. Regardless, the patch-pump system 100 may be coupled to a user's skin so the base surface 302 is adjacent to the user's skin. The patch-pump system 100 may be coupled to the user while the needle 206 is in the retracted position 400. Further, the reservoir 102 may have at least some fluid therein ready to be administered to the user. Once the patch-pump system 100 is coupled to the user, the electronic assembly 106 may transition the needle 206 from the retracted position 400 to the extended position 401 with the inserter 104. This extends the distal tip of the needle 206 subcutaneously into the user. The inserter 104 may initiate the insertion process through a user input to the electronic assembly 106 or directly to the motor 502. Alternatively, the inserter may be manually engaged to transition the needle 206 to the extended position 401. Once the needle 206 is in the extended position, the electronic assembly 106 may selectively engage the motor and gear assembly 204 to provide a set dose of the fluid in the reservoir 102 to the user through the needle 206 by selectively actuating the piston 202.

Once the user is prepared to remove the patch-pump system 100, the inserter 102 may transition the needle 206 to the retracted position 400 either through the electronic assembly 106 or by manually engaging the inserter 102. Once the needle 206 is in the retracted configuration 400, the adhesive holding the patch pump 100 to the user may be removed or the patch pump may be otherwise peeled from the user's skin.

While embodiments incorporating the principles of the present disclosure have been described hereinabove, the present disclosure is not limited to the described embodiments. Instead, this application is intended to cover any variations, uses, or adaptations of the disclosure using its general principles. Further, this application is intended to cover such departures from the present disclosure as come within known or customary practice in the art to which this disclosure pertains and which fall within the limits of the appended claims.

1-25: (canceled)

26. A infusion pump adherable to skin, comprising:
a driven needle inserter comprising a fluid channel therein along a rotation axis;
a replaceable fluid reservoir configured to releasable engage the driven needle inserter in fluid connection with the fluid channel;
a needle configured to extend outside the infusion pump and retract within the infusion pump while being fluidly coupled to the replaceable fluid reservoir by the fluid channel, the needle being substantially housed within the driven needle inserter in a retracted position; and
a controller configured to, upon user input,
instruct a motor to rotate the driven needle inserter to extend the needle outside the infusion pump subcutaneously into the skin of the user, and
instruct, once the needle is in an extended position, a pump motor to provide a set dose of fluid from the replaceable fluid reservoir to the user through the needle.

27. The infusion pump of claim 26, further comprising a base surface configured to be adhered to the skin of a user and wherein the needle is configured for insertion at an angle relative to a base plane defined by the base surface.

28. The infusion pump of claim 27, wherein the controller selectively controls an insertion angle of the needle relative to the base plane.

29. The infusion pump of claim 28, wherein the driven needle inserter is pivotal within the infusion pump to alter the insertion angle of the needle relative to the base plane.

30. The infusion pump of claim 26, wherein the controller is configured to control a degree of rotation of an inner part of the driven needle inserter coupled to the needle and therefore alter an extended depth of the needle within the user's skin.

31. The infusion pump of claim 26, wherein the controller is configured to selectively control an extension length and insertion angle of the needle.

32. The infusion pump of claim 26, wherein the replaceable fluid reservoir is configured to slide into and out of fluid coupling with the driven needle inserter and wherein the replaceable fluid reservoir comprises a latch configured to lock the replaceable fluid reservoir into a fluidly coupled position with the driven needle inserter.

33. The infusion pump of claim 26, wherein the controller is further configured to instruct the driven needle inserter to retract the needle from the skin of the user once the user is prepared to remove the infusion pump from their skin.

34. The infusion pump of claim 26, wherein the rotation axis is parallel to a base surface of the infusion pump and the driven needle inserter is configured to extend the needle to the extended position by rotating about the rotation axis.

35. The infusion pump of claim 34, wherein, the driven needle inserter comprises an inner part that rotates about the rotation axis that is substantially parallel to the base surface of the infusion pump to transition the needle from the retracted position to the extended position.

36. The infusion pump of claim 26, wherein the set dose comprises a predetermined dose rate.

37. The infusion pump of claim 26, wherein the needle is elastically deformed to fit within the driven needle inserter and bend along an inner surface of a housing of the infusion pump in the retracted position and maintains a substantially linear configuration as it extends outside of a base surface of the infusion pump.

38. The infusion pump of claim 37, further comprising a sleeve configured to extend along the needle to ensure the needle extends past the base surface in a substantially linear configuration, the sleeve comprising a spring positioned axially about the needle between the sleeve and the driven needle inserter.

39. The infusion pump of claim 38, wherein when an insertion angle is about ninety degrees, the sleeve compresses the spring and is positioned substantially within a cavity of the driven needle inserter and wherein when the insertion angle is less than ninety degrees, the sleeve at least partially extends outside of the cavity as the spring provides a bias towards the base surface thereby providing a channel for the needle to be further inserted into the skin of the user.

40. The infusion pump of claim 37, wherein the base surface is configured to adhere a user's skin until the user is prepared to remove the infusion pump.

41. The infusion pump of claim 26, wherein the infusion pump is a miniature infusion pump comprising a width of less than forty-nine millimeters wide, a depth of less than thirty-nine millimeters deep and a thickness of less than twelve millimeters.

42. The infusion pump of claim 26, further comprising a filter positioned between the replaceable fluid reservoir and a distal tip of the needle, wherein the filter at least partially filters one or more of phenol and meta-cresol residuals from fluid passing therethrough.

43. The infusion pump of claim 26, further comprising a first one-way valve positioned along the fluid channel between the replaceable fluid reservoir and a filter positioned in-line with the fluid channel between the replaceable fluid reservoir and a distal tip of the needle and a second one-way valve positioned along the fluid channel between a distal end of the needle and the filter.
